(19) United States
(12) Patent Application Publication
(10) Pub. No.: US 2022/0183904 A1
(43) Pub. Date: Jun. 16, 2022

(54) Adult Incontinence Garment Assembly

(71) Applicant: Ikechukwu Okpara, Madison, WI (US)

(72) Inventor: Ikechukwu Okpara, Madison, WI (US)

(21) Appl. No.: 17/117,199

(22) Filed: Dec. 10, 2020

(51) Int. Cl. A61F 13/49; A61F 13/56

(52) U.S. Cl. CPC A61F 13/49; A61F 13/49009; A61F 2013/49098; A61F 2013/49074; A61F 13/5633

(57) ABSTRACT

A adult incontinence garment assembly for assisting with incontinence and inhibiting bedsores includes a diaper that can be worn on a user thereby facilitating the diaper cover the user's crotch. A pad is integrated into the diaper to absorb bodily fluids from the user when the diaper is worn. A plurality of cushions is each integrated into the diaper and each of the cushions is strategically positioned on the diaper. In this way each of the cushions can be aligned with the user's hips and the user's tailbone when the diaper is worn. Additionally, each of the cushions is comprised of a resiliently compressible material to inhibit the user from developing bedsores while wearing the diaper.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to incontinence devices and more particularly pertains to a new incontinence device for assisting with incontinence and inhibiting bedsores. The incontinence device includes a plurality of cushions that are strategically positioned to reduce pressure on the user's hips and tailbone when the user is lying down or seated. In this way the cushions inhibit the development of bedsores if the user is bedridden.

### (2) Description of Related Art Including Information Disclosed Under 37 CFR 1.97 and 1.98

The prior art relates to incontinence devices including an incontinence diaper that includes a modular cushion that can be positioned in the diaper for inhibiting bedsores. The prior art also discloses a variety of diapers for absorbing bodily fluids. The prior art discloses a bedsore garment that includes a plurality of stimulation devices being integrated therein for stimulating blood flow in a user to inhibit the development of bedsores. Additionally, the prior art discloses a diaper that has inflatable bladders being integrated therein to inhibit the development of bedsores.

### BRIEF SUMMARY OF THE INVENTION

An embodiment of the disclosure meets the needs presented above by generally comprising a diaper that can be worn on a user thereby facilitating the diaper cover the user's crotch. A pad is integrated into the diaper to absorb bodily fluids from the user when the diaper is worn. A plurality of cushions is each integrated into the diaper and each of the cushions is strategically positioned on the diaper. In this way each of the cushions can be aligned with the user's hips and the user's tailbone when the diaper is worn. Additionally, each of the cushions is comprised of a resiliently compressible material to inhibit the user from developing bedsores while wearing the diaper.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an adult incontinence garment assembly according to an embodiment of the disclosure.

FIG. 2 is a front view of an embodiment of the disclosure.

FIG. 3 is a back view of an embodiment of the disclosure.

FIG. 4 is a perspective in-use view of an embodiment of the disclosure.

FIG. 5 is a right side view of an embodiment of the disclosure.

FIG. 6 is a cross sectional view taken along line 6-6 of FIG. 5 of an embodiment of the disclosure.

FIG. 7 is a front view of an alternative embodiment of the disclosure.

FIG. 8 is a back view of an alternative embodiment of the disclosure.

### DETAILED DESCRIPTION OF THE INVENTION

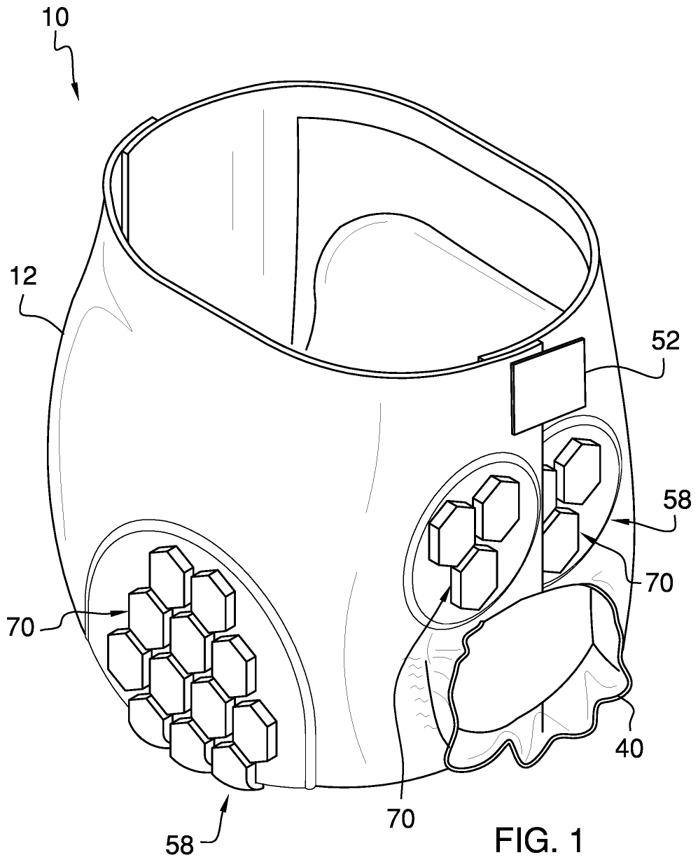

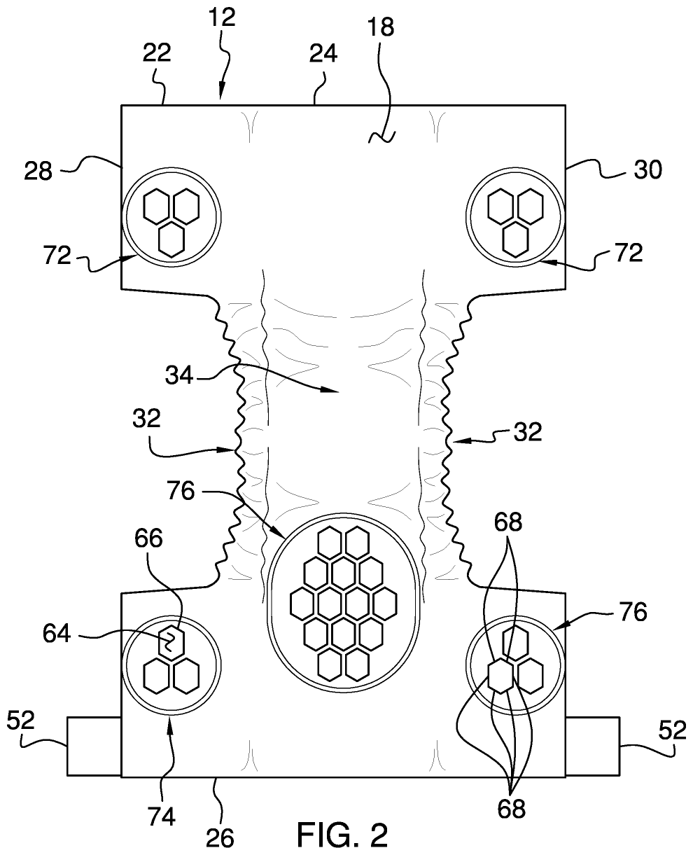

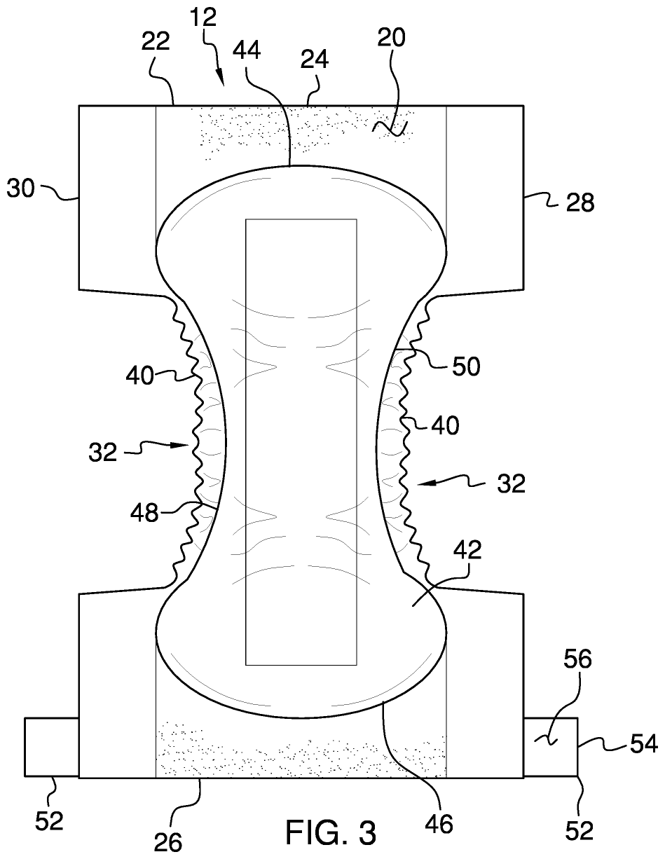

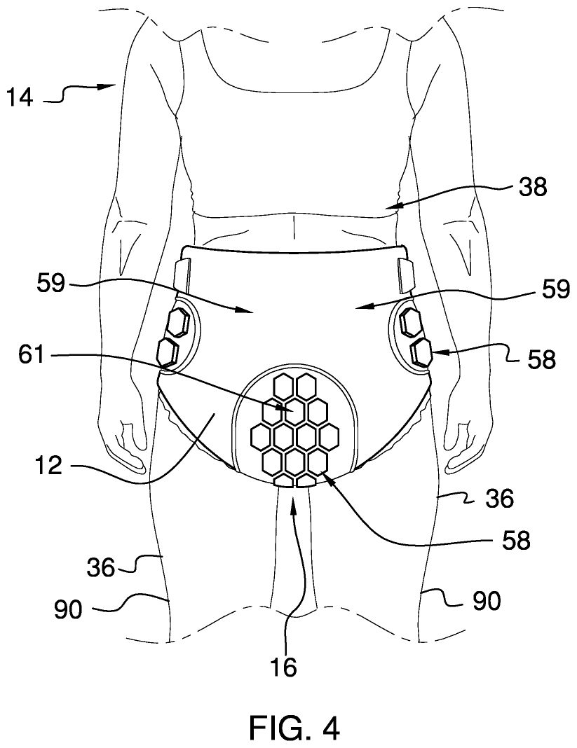

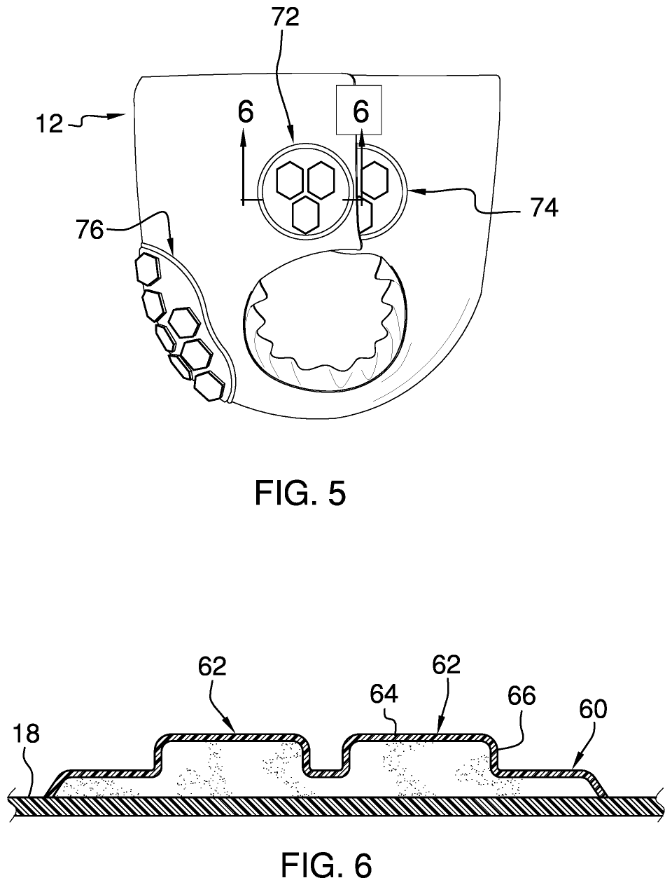

With reference now to the drawings, and in particular to FIGS. 1 through 8 thereof, a new incontinence device embodying the principles and concepts of an embodiment of the disclosure and generally designated by the reference numeral 10 will be described.

As best illustrated in FIGS. 1 through 8, the adult incontinence garment assembly 10 generally comprises a diaper 12 that can be worn on a user 14 thereby facilitating the diaper 12 cover the user's crotch 16. The diaper 12 has a front surface 18, a back surface 20 and a perimeter edge 22 extending therebetween, and the perimeter edge 22 has a front side 24, a back side 26, a first lateral side 28 and a second lateral side 30. Each of the first lateral side 28 and the second lateral side 30 has an arcuate portion 32 curving inwardly toward a middle 34 of the diaper 12 such that the diaper 12 has an hour glass shape. In this way the arcuate portion 32 of each of the first lateral side 28 and the second lateral side 30 extends around a respective one of the user's thighs 36 when the diaper 12 is worn having the front side 24 and the back side 26 is aligned with the user's waist 38. Additionally, the arcuate portion 32 of each of the first lateral side 28 and the second lateral side 30 is spaced from each of the front side 24 and the back side 26. The diaper 12 may be comprised of a fluid impermeable material to inhibit bodily fluids from passing through the diaper 12.

A pair of elastomeric bands 40 is provided and each of the elastomeric bands 40 is integrated into the diaper 12. Each of the elastomeric bands 40 is aligned with a respective one of the first lateral side 28 and the second lateral side 30 of the perimeter edge 22 of the diaper 12. In this way each of the elastomeric bands 40 encloses each of the first lateral side 28 and the second lateral side 30 around the respective user's thighs 36 when the diaper 12 is worn. Thus, the elastomeric bands 40 inhibit bodily fluids from leaking outwardly around the user's thighs 36.

A pad 42 is integrated into the diaper 12 and the pad 42 is comprised of a fluid absorbent material. In this way the pad 42 can absorb bodily fluids from the user 14 when the diaper 12 is worn. The pad 42 is positioned on the back surface 20 of the diaper 12 such that the pad 42 rests against the user 14 when diaper 12 is worn. The pad 42 has a first end 44, a second end 46, a first sidelong edge 48 and a second sidelong edge 50. Each of the first end 44 and the second end 46 is rounded, and each of the first sidelong edge 48 and the second sidelong edge 50 curves inwardly toward a middle 51 of the pad 42 between the first end 44 and the second end 46. Moreover, each of the first sidelong edge 48 and the second sidelong edge 50 is aligned with a respective one of the first lateral side 28 and the second lateral side 30 of the perimeter edge 22 the diaper 12. The first end 44 is spaced from the front side 24 of the perimeter edge 22 of the diaper 12 and the second end 46 is spaced from the back side 26 of the perimeter edge 22 of the diaper 12.

A pair of tabs 52 is each of the tabs 52 is coupled to and extends away from the diaper 12. Each of the tabs 52 is positioned on opposite sides of the diaper 12 from each other such that each of the tabs 52 extends partially around the user's waist 38 when the diaper 12 is worn. Each of the tabs 52 releasably engages the diaper 12 to retain the diaper 12 on the user 14. Each of the tabs 52 extends away from a respective one of the first lateral side 28 and the second lateral side 30 of the perimeter edge 22 of the diaper 12. Each of the tabs 52 is positioned adjacent to the front side 24 of the perimeter edge 22, and each of the tabs 52 has a distal end 54 and a first surface 56. The first surface 56 of each of the tabs 52 is comprised of an adhesive material thereby facilitating the first surface 56 of each of the tabs 52 to adhere to the front surface 18 of the diaper 12 adjacent to the back side 26 of the perimeter edge 22 when the diaper 12 is worn.

A plurality of cushions 58 is provided and each of the cushions 58 is integrated into the diaper 12. Each of the cushions 58 is strategically positioned on the diaper 12 such that each of the cushions 58 is aligned with the user's hips 78 and the user's tailbone 59 when the diaper 12 is worn. Moreover, each of the cushions 58 is comprised of a resiliently compressible material to inhibit the user 14 from developing bedsores while wearing the diaper 12.

Each of the cushions 58 is positioned on the front surface 18 of the diaper 12 each of the cushions 58 includes a panel portion 60 that is attached to the front surface 18 of the diaper 12 and a plurality of raised portion 62 extending away from the panel portion 60. Each of the raised portion 62 has a distal surface 64 with respect to the panel portion 60 and each of the raised portion 62 has a perimeter surface 66 extending between the distal surface 64 and the panel portion 60. The perimeter surface 66 of each of the raised portion 62 has a plurality of intersecting sides 68 such that each of the raised portion 62 has a hexagonal shape.

The plurality of cushions 58 comprises a set of clusters 70 of the cushions 58, and the set of clusters 70 includes a pair of first hip clusters 72, a pair of second hip clusters 74 and a tailbone cluster 76. Each of the first hip clusters 72 is positioned adjacent to an intersection between the front side 24 of the perimeter edge 22 of the diaper 12 and a respective one of the first lateral side 28 and the second lateral side 30 of the perimeter edge 22. In this way each of the first hip clusters 72 is aligned with a respective one of the user's hips 78 when the diaper 12 is worn. Each of the second hip clusters 74 is positioned adjacent to an intersection between the back side 26 of the perimeter edge 22 of the diaper 12 and a respective one of the first lateral side 28 and the second lateral side 30 of the perimeter edge 22. In this way each of the second hip clusters 74 is aligned with a respective one of the user's hips 78. Moreover, each of the first hip clusters 72 overlaps a respective one of the second hip clusters 74 when the diaper 12 is worn. In this way each of the first hip clusters 72 and the second hip clusters 74 can fully cover each of the user's hips 78.

The tailbone cluster 76 is centrally positioned between the first lateral side 28 and the second lateral side 30 of the perimeter edge 22 of the diaper 12. Additionally, the tailbone cluster 76 is spaced a strategic distance from the back side 26 of the perimeter edge 22. In this way the tailbone cluster 76 is aligned with the user's tailbone 59 when the diaper 12 is worn.

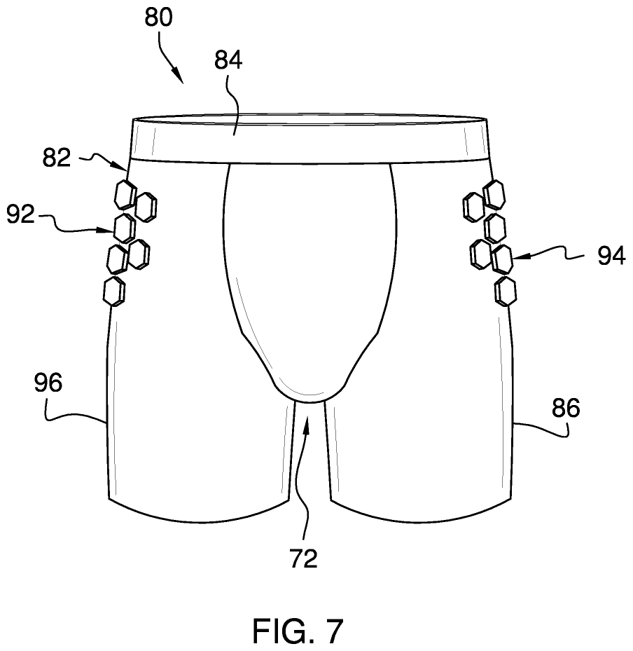

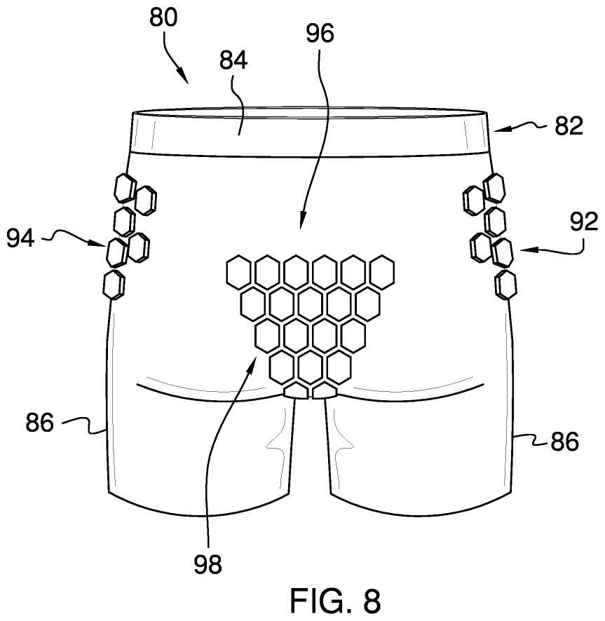

In an alternative embodiment 80 as is shown in FIGS. 7 and 8, the diaper 12 is structured as a pair of shorts 82 that includes a waist band 84 and a pair of legs 86 each extending downwardly from a crotch 88 of the diaper 12. In this way each of the legs 86 accommodates a respective one of the user's legs 90 when the diaper 12 is worn having the crotch 88 extending across the user's crotch 16. The plurality of cushions 58 includes a set of first hip cushions 92, a set of second hip cushions 94 and a set of tailbone cushions 96. Each of the first hip cushions 92 and the second hip cushions 94 is positioned on a respective one of the user's legs 90 at a location that is positioned adjacent to the waist band 84. Additionally, each of the tailbone cushions 96 is positioned on a back side 98 of the crotch 88 and each of the tailbone cushions 58 is aligned with the user's tailbone 59 when the diaper 12 is worn.

In use, the diaper 12 is worn on the user 14 to assist the user 14 with incontinence or when the user 14 will not have access to a bathroom for an extended period of time. The pad 42 absorbs bodily fluids, such as urine, while the user 14 is wearing the diaper 12. Additionally, each of the cushions 58 relieves pressure on the user's hips 78 and the user's tailbone 59 while the user 14 is lying down or sitting. In this way the cushions 58 inhibit the user 14 from developing bedsores or other pressure related sores that are common in bedridden users 14. The diaper 12 is removed when the pad 42 becomes soaked with the bodily fluids and the diaper 12 is discarded.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An adult incontinence garment assembly for absorbing bodily fluid and inhibiting the development of bed sores, said assembly comprising:
a diaper being configured to be worn on a user thereby facilitating the diaper cover the user's crotch;
a pad being integrated into said diaper, said pad being comprised of a fluid absorbent material wherein said pad is configured to absorb bodily fluids from the user when said diaper is worn;
a pair of tabs, each of said tabs being coupled to and extending away from said diaper, each of said tabs being positioned on opposite sides of said diaper from each other wherein each of said tabs is configured to extend partially around the user's waist when said diaper is worn, each of said tabs releasably engaging said diaper wherein said tabs are configured to retain said diaper on the user; and
a plurality of cushions, each of said cushions being integrated into said diaper, each of said cushions being strategically positioned on said diaper wherein each of said cushions is configured to be aligned with the user's hips and the user's tailbone when said diaper is worn, each of said cushions being comprised of a resiliently compressible material wherein each of said cushions is configured to inhibit the user from developing bedsores while wearing said diaper.

2. The assembly according to claim 1, wherein said diaper has a front surface, back surface and a perimeter edge extending therebetween, said perimeter edge having a front side, a back side, a first lateral side and a second lateral side, each of said first lateral side and said second lateral side having an arcuate portion curving inwardly toward a middle of said diaper such that said diaper has an hour glass shape wherein said arcuate portion of each of said first lateral side and said second lateral side is configured to extend around a respective one of the user's thighs when said diaper is worn having said front side and said back side being aligned with the user's waist, said arcuate portion of each of said first lateral side and said second lateral side being spaced from each of said front side and said back side.

3. The assembly according to claim 2, further comprising a pair of elastomeric bands, each of said elastomeric bands being integrated into said diaper, each of said elastomeric bands being aligned with a respective one of said first lateral side and said second lateral side of said perimeter edge of said diaper wherein each of said elastomeric bands is configured to enclose each of said first lateral side and said second lateral side around the respective user's thighs when said diaper is worn to inhibit bodily fluids from leaking outwardly around the user's thighs.

4. The assembly according to claim 2, wherein said pad is positioned on said back surface of said diaper wherein said pad is configured to rest against the user when diaper is worn, said pad having a first end, a second end, a first sidelong edge and a second sidelong edge, each of said first end and said second end being rounded, each of said first sidelong edge and said second sidelong edge curving inwardly toward a middle of said pad between said first end and said second end.

5. The assembly according to claim 4, wherein each of said first sidelong edge and said second sidelong edge is aligned with a respective one of said first lateral side and said second lateral side of said diaper, said first end being spaced from said front side of said perimeter edge of said diaper, said second end being spaced from said back side of said perimeter edge of said diaper.

6. The assembly according to claim 2, wherein each of said tabs extends away from a respective one of said first lateral side and said second lateral side of said perimeter edge of said diaper, each of said tabs being positioned adjacent to said front side of said perimeter edge, each of said tabs having a distal end and a first surface.

7. The assembly according to claim 6, wherein said first surface of each of said tabs is comprised of an adhesive material thereby facilitating said first surface of each of said tabs to adhere to said front surface of said diaper adjacent to said back side of said perimeter edge when said diaper is worn.

8. The assembly according to claim 2, wherein each of said cushions is positioned on said front surface of said diaper, each of said cushions having a distal surface with respect to said front surface said diaper, each of said cushions having a perimeter surface extending between said distal surface and said front surface, said perimeter surface of each of said cushions having a plurality of intersecting sides such that each of said cushions having a hexagonal shape.

9. The assembly according to claim 2, wherein said plurality of cushions comprises a set of clusters of said cushions, said set of clusters including a pair of first hip clusters, a pair of second hip clusters and a tailbone cluster.

10. The assembly according to claim 9, wherein each of said first hip clusters is positioned adjacent to an intersection between said front side of said perimeter edge of said diaper and a respective one of said first lateral side and said second lateral side of said perimeter edge wherein each of said first hip clusters is configured to be aligned with a respective one of the user's hips when said diaper is worn.

11. The assembly according to claim 9, wherein each of said second hip clusters is positioned adjacent to an intersection between said back side of said perimeter edge of said diaper and a respective one of said first lateral side and said second lateral side of said perimeter edge wherein each of said second hip clusters is configured to be aligned with a respective one of the user's hips.

12. The assembly according to claim 9, wherein each of said first hip clusters overlaps a respective one of said second hip clusters when said diaper is worn wherein each of said first hip clusters and said second hip clusters is configured to fully cover each of the user's hips.

13. The assembly according to claim 9, wherein said tailbone cluster is centrally positioned between said first lateral side and said second lateral side of said perimeter edge of said diaper, said tailbone cluster being spaced a strategic distance from said back side of said perimeter edge wherein said tailbone cluster is configured to be aligned with the user's tailbone when said diaper is worn.

14. An adult incontinence garment assembly for absorbing bodily fluid and inhibiting the development of bed sores, said assembly comprising:
a diaper being configured to be worn on a user thereby facilitating the diaper cover the user's crotch, said diaper having a front surface, back surface and a perimeter edge extending therebetween, said perimeter edge having a front side, a back side, a first lateral side and a second lateral side, each of said first lateral side and said second lateral side having an arcuate portion curving inwardly toward a middle of said diaper such that said diaper has an hour glass shape wherein said arcuate portion of each of said first lateral side and said second lateral side is configured to extend around a respective one of the user's thighs when said diaper is worn having said front side and said back side being aligned with the user's waist, said arcuate portion of each of said first lateral side and said second lateral side being spaced from each of said front side and said back side;
a pair of elastomeric bands, each of said elastomeric bands being integrated into said diaper, each of said elastomeric bands being aligned with a respective one of said first lateral side and said second lateral side of said perimeter edge of said diaper wherein each of said elastomeric bands is configured to enclose each of said first lateral side and said second lateral side around the respective user's thighs when said diaper is worn to inhibit bodily fluids from leaking outwardly around the user's thighs;
a pad being integrated into said diaper, said pad being comprised of a fluid absorbent material wherein said pad is configured to absorb bodily fluids from the user when said diaper is worn, said pad being positioned on said back surface of said diaper wherein said pad is configured to rest against the user when diaper is worn, said pad having a first end, a second end, a first sidelong edge and a second sidelong edge, each of said first end and said second end being rounded, each of said first sidelong edge and said second sidelong edge curving inwardly toward a middle of said pad between said first end and said second end, each of said first sidelong edge and said second sidelong edge being aligned with a respective one of said first lateral side and said second lateral side of said diaper, said first end being spaced from said front side of said perimeter edge of said diaper, said second end being spaced from said back side of said perimeter edge of said diaper;
a pair of tabs, each of said tabs being coupled to and extending away from said diaper, each of said tabs being positioned on opposite sides of said diaper from each other wherein each of said tabs is configured to extend partially around the user's waist when said diaper is worn, each of said tabs releasably engaging said diaper wherein said tabs are configured to retain said diaper on the user, each of said tabs extending away from a respective one of said first lateral side and said second lateral side of said perimeter edge of said diaper, each of said tabs being positioned adjacent to said front side of said perimeter edge, each of said tabs having a distal end and a first surface, said first surface of each of said tabs being comprised of an adhesive material thereby facilitating said first surface of each of said tabs to adhere to said front surface of said diaper adjacent to said back side of said perimeter edge when said diaper is worn; and
a plurality of cushions, each of said cushions being integrated into said diaper, each of said cushions being strategically positioned on said diaper wherein each of said cushions is configured to be aligned with the user's hips and the user's tailbone when said diaper is worn, each of said cushions being comprised of a resiliently compressible material wherein each of said cushions is configured to inhibit the user from developing bedsores while wearing said diaper, each of said cushions being positioned on said front surface of said diaper, each of said cushions having a distal surface with respect to said front surface said diaper, each of said cushions having a perimeter surface extending between said distal surface and said front surface, said perimeter surface of each of said cushions having a plurality of intersecting sides such that each of said cushions having a hexagonal shape, said plurality of cushions comprising a set of clusters of said cushions, said set of clusters including a pair of first hip clusters, a pair of second hip clusters and a tailbone cluster, each of said first hip clusters being positioned adjacent to an intersection between said front side of said perimeter edge of said diaper and a respective one of said first lateral side and said second lateral side of said perimeter edge wherein each of said first hip clusters is configured to be aligned with a respective one of the user's hips when said diaper is worn, each of said second hip clusters being positioned adjacent to an intersection between said back side of said perimeter edge of said diaper and a respective one of said first lateral side and said second lateral side of said perimeter edge wherein each of said second hip clusters is configured to be aligned with a respective one of the user's hips, each of said first hip clusters overlapping a respective one of said second hip clusters when said diaper is worn wherein each of said first hip clusters and said second hip clusters is configured to fully cover each of the user's hips, said tailbone cluster being centrally positioned between said first lateral side and said second lateral side of said perimeter edge of said diaper, said tailbone cluster being spaced a strategic distance from said back side of said perimeter edge wherein said tailbone cluster is configured to be aligned with the user's tailbone when said diaper is worn.

15. The assembly according to claim 14, wherein said diaper comprises a waist band and a pair of legs each extending downwardly from a crotch of said diaper wherein each of said legs is configured to have a respective one of the user's legs extended therethrough when said diaper is worn having said crotch extending across the user's crotch.

16. The assembly according to claim 15, wherein said plurality of cushions includes a set of first hip cushions, a set of second hip cushions and a set of tailbone cushions, each of said first hip cushions and said second hip cushions being positioned on a respective one of said legs at a location being positioned adjacent to said waist band, each of said tailbone cushions being positioned on a back side of said crotch portion wherein each of said tailbone cushions is configured to be aligned with the user's tailbone when said diaper is worn.
